(19) United States
(12) Patent Application Publication
(10) Pub. No.: US 2021/0077933 A1
(43) Pub. Date: Mar. 18, 2021

(54) Filtered Enclosure for Cell Culture Samples for Incubators

(71) Applicant: Mott Corporation, Farmington, CT (US)

(72) Inventor: Gregory Tedeschi, Cromwell, CT (US)

(21) Appl. No.: 17/025,352

(22) Filed: Sep. 18, 2020

Related U.S. Application Data
(60) Provisional application No. 62/902,204, filed on Sep. 18, 2019.

(51) Int. Cl. B01D 46/00; B01D 39/10; B01D 46/10; C12M 1/00; C12M 3/00

(52) U.S. Cl. CPC B01D 46/0028; B01D 39/10; B01D 46/10; B01D 2239/0442; C12M 23/44; B01D 2239/0478; B01D 2239/1216; C12M 41/14

(57) ABSTRACT

Described herein is a filtered, reusable, autoclavable cell culture sample container that would enclose a cell culture growth container such as a flask, Petri dish or well plate and it would be stored in an incubator with or without adjacent samples and sample containers. The purpose of the product is to provide a sterile incubation environment with free-flowing gas exchange with the surrounding environment.

### CROSS-REFERENCE TO RELATED PATENT APPLICATION

This patent application claims a benefit to the Sep. 18, 2019 filing date of U.S. Provisional Patent Application Ser. No. 62/902,204, titled “Filtered Enclosure for Cell Culture Samples for Incubators.” The disclosure of U.S. 62/902,204 is incorporated by reference herein in its entirety.

### BACKGROUND

Current cell culture incubation utilizes a variety of incubator chambers which are usually configured to regulate heat, humidity and gas concentration to promote cell growth. Multiple samples are stored together in the incubator chamber without isolation from one another. Typically, multiple users store samples in the same incubator chamber and run a risk of cross-contamination or carry over contaminants from adjacent samples.

Cell culture contamination is a pain point of cell biologists and by some estimates accounts for 50% of all contamination issues in bioprocessing. It has been noted that mycoplasma infestations are widespread and costing laboratories millions of dollars in lost research.

One recent design is a line of incubator chambers which includes a post-office box design of individual containers each with their own incubation slot within a larger chamber. However, this design is complex and expensive.

### BRIEF SUMMARY

Disclosed herein is enclosure useful as an incubation chamber for cell cultures. The chamber has a top, a bottom and a plurality of sidewalls spacing the top from the bottom and defining an internal chamber for receiving the cell cultures. A portion of one or more of the top, the bottom and the plurality of sidewalls is formed from a porous metal filtration media.

### BRIEF DESCRIPTION OF THE DRAWINGS

FIG. 1 illustrates an incubator chamber for a cell culture in accordance with a first embodiment disclosed herein.

FIG. 2 illustrates the incubator chamber of FIG. 1 further including stackable mid-sections to increase container height.

FIG. 3 illustrates an incubator chamber for a cell culture in accordance with a second embodiment disclosed herein.

FIG. 4 illustrates the incubator chamber of FIG. 3 in cross-section.

FIG. 5 illustrates a slidable drawer as a component of the incubator chamber of FIG. 3.

FIG. 6 is a perspective view of the incubator chamber of FIG. 2.

### DETAILED DESCRIPTION

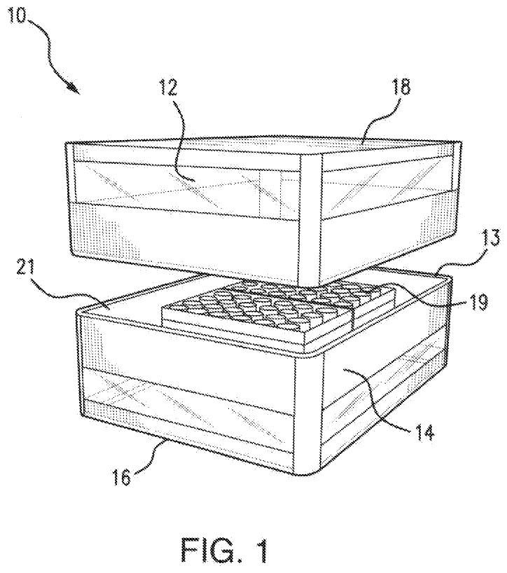

FIG. 1 illustrates an incubator chamber 10 that may be retrofit into various cell culture incubator models. The incubator chamber 10 is autoclavable and may include antimicrobial filters. The design is at least a 2-piece system having a base 16 and a lid 18. Storage capacity may be increased by the inclusion of one or more stackable mid sections to increase container height.

The incubator chamber 10 includes at least a two-piece compression fitting enclosure with a transparent first portion 12 formed from glass or autoclavable plastic for sample visualization. A compressible material 13, is formed from polytetrafluoroethylene (PTFE) or another high temperature withstanding, bio-inert, polymer. The compressible material 13 ensures a hermetic seal where a hermetic seal is defined as a leak rate of less than 10⁻⁸ mbar/second at a temperature of 37 degrees centigrade/

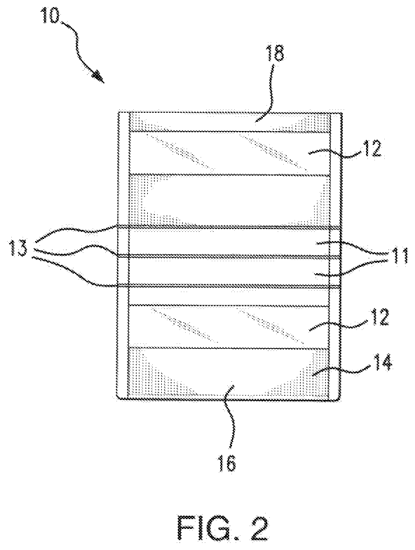

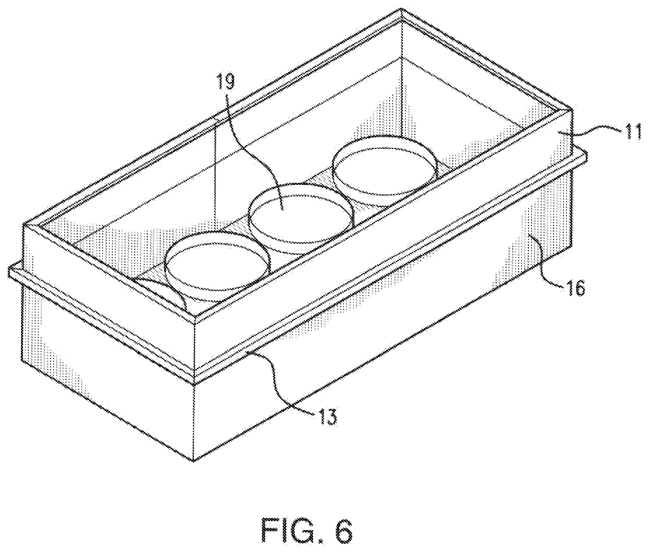

A second portion 14 of the incubator chamber 10 is formed from a porous metal filtration media, such as stainless steel or copper and is effective to mitigate sample contamination. If not formed from copper, the second portion 14 porous metal filtration media may be plated with copper as an antimicrobial agent. As shown in FIGS. 2 and 6, intermediate stackable pieces 11 may be used to adjust the height of the incubator chamber 10 to accommodate more samples 19. Typically, the intermediate stackable pieces 11 are formed from porous copper or stainless steel and compressible material 13, such as PTFE, is disposed between the intermediate stackable pieces as well as between those pieces and the lid 18 and base 16 to maintain hermeticity.

Preferably, at least part of the lid 18 is formed from the transparent first portion 12 to facilitate visual observation of enclosed samples 19. Users benefit by being able to see the enclosed samples 19 when transporting to ensure that the enclosed samples 19 are not shifting or leaking during transport.

The second portion 14 is a porous metal filtration media that is a sintered metal powder porous sheet having a nominal average sub-micron (less than 1×10⁻⁶ meters (<1 μm)) pore size. A preferred location for porous second portion 14 is along sides of the incubator chamber 10 where it would least obscure viewing of the enclosed samples 19 contained within the incubator chamber. Exemplary outer dimension for incubator chambers am nominally 5.5 inch×5.5 inch by 11 inch and 6 inch×9 inch×20 inch. Exemplary dimensions for the open interior portion 21 that receives the enclosed samples 19 are 5.25 inch×5.25 inch×10.75 inch and 5.75 inch×8.75 inch×19.75 inch.

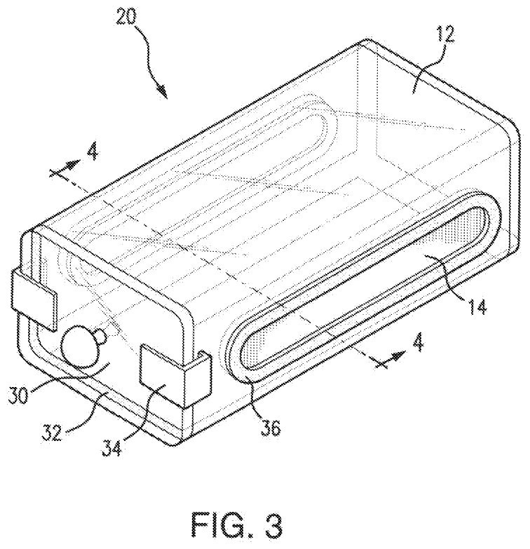

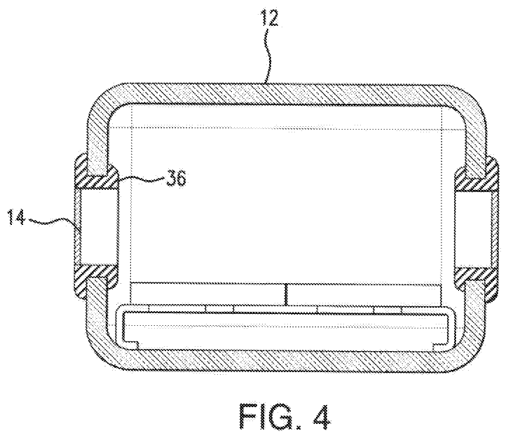

FIG. 3 illustrates an alternative incubator chamber 20 having porous metal filtration media second portion 14 forming a section of the chamber sidewalls while the top, bottom and remainder of the sidewalls are formed from the transparent first portion 12. Door 30 is removable to allow insertion and removal of samples. A hermetic seal is achieved by compressible gasket 32 and locking clamps 34. Compressible gasket 36 ensures a hermetic seal between the porous metal filtration portion 14 and transparent first portion 12. The cross-sectional view in FIG. 4 shows the compressible gasket 36 both supporting the porous metal filtration media 14 and providing a hermetic seal with the transparent first portion 12.

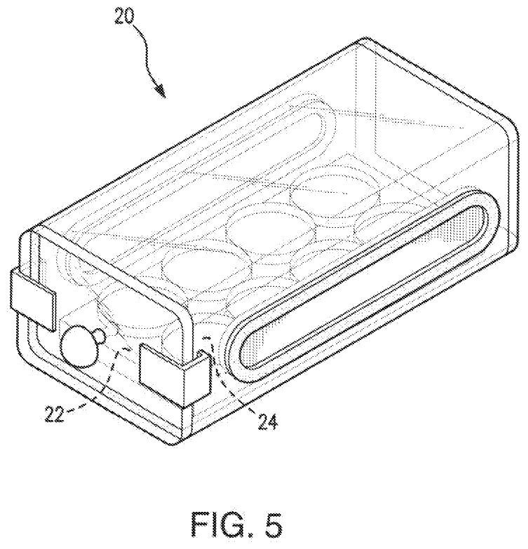

As seen in FIG. 5, the incubator chamber 20 may include a slidable drawer 22 to provide access to a plurality of cell culture growth containers 24, such as flasks, Petri dishes or well plates.

Basic elements both the incubator chamber 10 and the incubator chamber 20 include:
sub-micron (<1 μm) media grade sintered porous metal;
Reusable filtration portion is sterilizable, such as with peroxide gas;
Fully autoclavable (>121 C for 30 minutes);
Corrosion resistance to humid and peroxide gas environment;
Gas exchange to sustain cells, O₂ and CO₂ levels;
Antimicrobial & hydrophobic filter surface;
Complete sterilization of gas exchange through device; and
See-through (window) to sample visualization

I claim:

1. An enclosure, comprising:
a top;
a bottom; and
a plurality of sidewalls spacing the top from the bottom and defining an internal chamber;
wherein at least a first portion of one or more of the top, the bottom and the plurality of sidewalls is a porous metal filtration media.

2. The enclosure of claim 1 wherein at least a second portion of one or more of the top, the bottom and the plurality of sidewalls is optically transparent.

3. The enclosure of claim 2 having:
a first component including the top and a first portion of the plurality of sidewalls; and
a second component including the bottom and a second portion of the plurality of sidewalls;
wherein the first component and the second component engage one another forming the internal chamber.

4. The enclosure of claim 3 wherein the first component and the second component engage via a compression fitting.

5. The enclosure of claim 2 wherein at least a portion of one of the plurality of sidewalls is removable to form an opening.

6. The enclosure of claim 5 further including a slideable drawer that fits within the internal chamber and sized to fit through the opening.

7. The enclosure of claim 2 wherein the porous metal filtration media has a sub-micron (<1 μm) nominal average pore size.

8. The enclosure of claim 2 wherein the porous metal filtration media is copper or a copper-base alloy.

9. The enclosure of claim 2 wherein the porous metal filtration media is coated with copper or a copper-base alloy.

10. The enclosure of claim 9 wherein the porous metal filtration media is stainless steel.
